SEQUENCE LISTING (15 sequences)

SEQ ID NO: 1 - protein, 204 aa, Homo sapiens
MEESVVRPSVFVVDGQTDIPFTRLGRSHRRQSCSVARDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 2 - DNA, 615 bp, Homo sapiens
atggaggagagtgtcgtacggccctcagtgtttgtggtggatggacagaccgacatcccattcacgaggctgggacgaagccaccggagacagtcgtgcagtgtggcccgggacggacctgcaggctcctgggagcagctgatacaagagcgaaggtctcacgaggtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggagaaggtggtcgtccgtgtgctggatgaacgcctggttcgactgcgtgatggtacccggtcttacttcggggctttcatggtgtga

SEQ ID NO: 3 - protein, 239 aa, Mus musculus
MESVVQPSVFVVDGQTDIPFRRLEQNHRRRRCGTVQVSLALVLLLGAGLATQGWFLLRLHQRLGDIVAHLPDGGKGSWEKLIQDQRSHQANPAAHLTGANASLIGIGGPLLWETRLGLAFLRGLTYHDGALVTMEPGYYYVYSKVQLSGVGCPQGLANGLPITHGLYKRTSRYPKELELLVSRRSPCGRANSSRVWWDSSFLGGVVHLEAGEEVVVRVPGNRLVRPRDGTRSYFGAFMV

SEQ ID NO: 4 - DNA, 720 bp, Mus musculus
atggagagtgtggtacagccttcagtgtttgtggtggatggacagacggacatcccattcaggcggctggaacagaaccaccggagacggcgctgtggcactgtccaggtcagcctggccctggtgctgctgctaggtgctgggctggccactcagggctggtttctcctgagactgcatcaacgtcttggagacatagtagctcatctgccagatggaggcaaaggctcctgggagaagctgatacaagatcaacgatctcaccaggccaacccagcagcacatcttacaggagccaacgccagcttgataggtattggtggacctctgttatgggagacacgacttggcctggccttcttgaggggcttgacgtatcatgatggggccctggtgaccatggagcccggttactactatgtgtactccaaagtgcagctgagcggcgtgggctgcccccaggggctggccaatggcctccccatcacccatggactatacaagcgcacatcccgctacccgaaggagttagaactgctggtcagtcggcggtcaccctgtggccgggccaacagctcccgagtctggtgggacagcagcttcctgggcggcgtggtacatctggaggctggggaagaggtggtggtccgcgtgcctggaaaccgcctggtcagaccacgtgacggcaccaggtcctatttcggagctttcatggtctga

SEQ ID NO: 5 - protein, 8 aa, Homo sapiens
CRGRRSTG

SEQ ID NO: 6 - protein, 250 aa, Artificial Sequence
  Synthetic sequence
MESVVQPSVFVVDGQTDIPFRRLEQNHRRRRCGTVQVSLALVLLLGAGLATQGWFLLRLHQRLGDIVAHLPDGGKGSWEKLIQDQRSHQANPAAHLTGANASLIGIGGPLLWETRLGLAFLRGLTYHDGALVTMEPGYYYVYSKVQLSGVGCPQGLANGLPITHGLYKRTSRYPKELELLVSRRSPCGRANSSRVWWDSSFLGGVVHLEAGEEVVVRVPGNRLVRPRDGTRSYFGAFMVGGGCRGRRSTG

SEQ ID NO: 7 - protein, 5 aa, Homo sapiens
CGKRK

SEQ ID NO: 8 - protein, 5 aa, Homo sapiens
CREKA

SEQ ID NO: 9 - protein, 5 aa, Homo sapiens
SRPRR

SEQ ID NO: 10 - protein, 5 aa, Homo sapiens
CDTRL

SEQ ID NO: 11 - protein, 10 aa, Homo sapiens
PQRRSARLSA

SEQ ID NO: 12 - protein, 31 aa, Homo sapiens
KDEPQRRSARLSAKPAPPKPEPKPKKAPAKK

SEQ ID NO: 13 - protein, 9 aa, Homo sapiens
CGNKRTRGC

SEQ ID NO: 14 - DNA, 20 bp, Artificial Sequence
  Synthetic sequence
tccatgacgttcctgatgct

SEQ ID NO: 15 - protein, 6 aa, Homo sapiens
CNGRCG

(19) United States
(12) Patent Application Publication
(10) Pub. No.: US 2020/0000877 A1
(43) Pub. Date: Jan. 2, 2020

(54) TREATMENT OF TUMOURS USING PEPTIDE-PROTEIN CONJUGATES

(71) Applicant: The University of Western Australia, Crawley (AU)

(72) Inventors: Ruth Ganss, Nedlands (AU); Anna Johansson, Karrinyup (AU)

(21) Appl. No.: 16/538,516

(22) Filed: Aug. 12, 2019

Related U.S. Application Data
(63) Continuation of application No. 14/856,238, filed on Sep. 16, 2015.
(60) Provisional application No. 62/051,090, filed on Sep. 16, 2014.

(51) Int. Cl. A61K 38/17; A61N 5/10

(52) U.S. Cl. CPC A61K 38/177; A61N 5/10

(57) ABSTRACT

Provided herein are methods for modulating tumour stroma, normalizing tumour vasculature and/or improving vascular function in a tumour, comprising exposing a tumour to an effective amount of a peptide-protein conjugate comprising a LIGHT polypeptide and a tumour homing peptide. Also provided are methods for treating tumours and increasing the survival time of tumour-bearing patients, comprising administering an effective amount of a peptide-protein conjugate comprising a LIGHT polypeptide and a tumour homing peptide.

### CROSS-REFERENCE TO RELATED APPLICATIONS

This application is a continuation of and claims the benefit and priority to U.S. patent application Ser. No. 14/856,238, filed Sep. 16, 2015, which claims the benefit of priority to U.S. Provisional Application Ser. No. 62/051,091, filed Sep. 16, 2014. The entire contents of the above-application is hereby incorporated by reference and made a part of this specification. Any and all priority claims identified in the Application Data Sheet, or any correction thereto, are hereby incorporated by reference under 37 CFR 1.57.

### REFERENCE TO SEQUENCE LISTING

A Sequence Listing submitted as an ASCII text file via EFS-Web is hereby incorporated by reference in accordance with 35 U.S.C. § 1.52(e). The name of the ASCII text file for the Sequence Listing is SeqList-DAVI561-001C1.txt, the date of creation of the ASCII text file is Aug. 12, 2019, and the size of the ASCII text file is 10 KB.

### FIELD OF THE INVENTION

The present invention relates generally to methods and compositions for the treatment of tumours and for increasing the survival time of patients having tumours. Also provided are methods and compositions for modulating or normalizing the stroma and/or vasculature within a tumour and improving vascular function within a tumour. The present invention relates to uses of protein conjugates comprising a LIGHT polypeptide conjugated to a tumour-homing peptide, optionally as an adjunct to immunotherapy, chemotherapy and/or radiotherapy.

### BACKGROUND OF THE INVENTION

To obtain nutrients for their growth and to metastasize to distant organs, cancer cells co-opt the host vasculature, induce new vessel formation (angiogenesis), and recruit endothelial and other stromal cells from the bone marrow. The resulting vasculature within tumours is structurally and functionally abnormal. Tumour blood vessels are leaky and dilated leading to interstitial hypertension. Endothelial cells lining the vessels have aberrant morphology, and pericytes, that provide support for the endothelial cells, are loosely attached, immature or absent. The basement membrane is also often abnormal.

These structural and functional abnormalities in tumour vessels create abnormal tumour microenvironment with, for example, impaired oxygen and acidosis. This hypoxia in turn can promote tumour invasion, metastasis and malignancy. The abnormal tumour microenvironment caused by stromal cells including the irregularities in tumour vasculature can also impede the effective delivery of anti-cancer therapeutics, thereby reducing their efficacy.

In devising new treatments of tumours; much effort has focused on reducing or abolishing these vascular abnormalities using anti-angiogenic agents, which also temporarily normalize the tumour vasculature and alleviate hypoxia (see, for example, Jain, 2001 Nat. Med. 9, 685-693). However anti-angiogenic agents can cause extensive damage to, including destruction of, tumour vessels.

Accordingly, there is a need for novel treatments can that effectively target the tumour vasculature without causing the damaging effects of known anti-angiogenic therapies.

### SUMMARY OF THE INVENTION

A first aspect of the invention provides a method for modulating tumour stroma, normalizing tumour vasculature and/or improving vascular function in a tumour, the method comprising exposing a tumour to an effective amount of a peptide-protein conjugate comprising a LIGHT polypeptide (also known as TNFSF14) and a tumour homing peptide.

The LIGHT polypeptide may comprise the amino acid sequence set forth in SEQ ID NO:1 or be encoded by the nucleotide sequence set forth in SEQ ID NO:2.

The tumour homing peptide may be selected from, for example, an RGR-containing peptide, an NGR-containing peptide, an RGD-containing peptide, a CGKRK-containing peptide and a CREKA-containing peptide. In an exemplary embodiment the tumour homing peptide is an RGR-containing peptide. The RGR-containing peptide may comprise the amino acid sequence CRGRRSTG (SEQ ID NO:5). In an embodiment the RGR-containing peptide is conjugated at the C-terminal of the LIGHT polypeptide. The tumour homing peptide may be conjugated to the LIGHT polypeptide via a linker sequence. In exemplary embodiments the linker may comprise one or more, optionally two or more or three or more glycine (G) residues.

In an embodiment of the first aspect the effective amount of the LIGHT-RGR conjugate may be between about 0.2 ng and 20 ng per kg body weight. In one example, the effective amount may be about 6 ng per kg body weight.

Said normalization of tumour vasculature and/or improvement of tumour vessel function may comprise or be characterized by one or more of: change in secretion of factors/cytokines from stromal cells including endothelial cells, pericytes, fibroblasts, macrophages and other intratumoral immune cells, selective loss of large vessels; reduced leakiness of vessels; pericyte re-attachment to vessels; alignment of surrounding collagen IV fibres; enhanced infiltration of CD8+ and/or CD45+ T cells; increased expression of inflammatory adhesion molecules; and increased expression of vascular smooth muscle markers in a smooth muscle (αSMC)-positive tumour pericytes. In some embodiments, said normalization of tumour vasculature and/or improvement of tumour vessel function may comprise or result in one or more of restoration of tumour blood vessel integrity, a reduction in leakiness of tumour blood vessels and/or an increase in tumour perfusion.

Said normalization of tumour vasculature and/or improvement of tumour vessel function may effect or induce a reduction in edema formation associated with tumours, for example brain tumours. Said normalization of tumour vasculature and/or improvement of tumour vessel function may effect or induce a reduction in tumour metastatic spreading, in particular a reduction in blood borne tumour metastasis.

A second aspect of the invention provides a method for inducing the formation of ectopic or tertiary lymph nodes in a tumour, the method comprising exposing the tumour to an effective amount of a peptide-protein conjugate comprising a LIGHT polypeptide and a tumour homing peptide.

The ectopic or tertiary lymph nodes may comprise high endothelial venules (HEVs).

The LIGHT polypeptide may comprise the amino acid sequence set forth in SEQ ID NO:1 or be encoded by the nucleotide sequence set forth in SEQ ID NO:2.

In an exemplary embodiment the tumour homing peptide is an RGR-containing peptide. The RGR-containing peptide may comprise the amino acid sequence CRGRRSTG (SEQ ID NO:5). In an embodiment the RGR-containing peptide is conjugated at the C-terminal of the LIGHT polypeptide. The tumour homing peptide may be conjugated to the LIGHT polypeptide via a linker sequence. In exemplary embodiments the linker may comprise one or more, optionally two or more or three or more glycine (G) residues.

In an embodiment of the second aspect the effective amount of the LIGHT-RGR conjugate may be between about 20 ng and 2000 ng per kg body weight. In one example, the effective amount may be about 600 to 700 ng per kg body weight.

A third aspect of the invention provides a method for treating a tumour in a subject, the method comprising administering to the subject an effective amount of a peptide-protein conjugate comprising a LIGHT polypeptide and a tumour homing peptide as disclosed herein.

The peptide-protein conjugate may be administered to the subject in combination with chemotherapy, immunotherapy and/or radiotherapy. The conjugate may be administered to the subject prior to, concomitantly with, or subsequent to the chemotherapy, immunotherapy and/or radiotherapy.

Immunotherapy may comprise adoptive cell transfer or the administration of one or more anti-tumour or immune checkpoint inhibitors, tumour-specific vaccines or other immune cell modulating agents optionally with, for example, autologous tumour material or known anti-tumour antigen/adjuvant formulations. Adoptive cell transfer may comprise the transfer of autologous tumour infiltrating lymphocytes. In exemplary embodiments, the immune checkpoint inhibitor may comprise anti-CTLA4 antibodies or anti-PD-1 antibodies. In exemplary embodiments the chemotherapy may comprise administration of cyclophosphamide.

In a particular embodiment the method comprises administration of the LIGHT polypeptide conjugated to a tumour homing peptide, optionally an RGR-containing peptide, in combination with one or more immune checkpoint inhibitors. The one or more immune checkpoint inhibitors may comprise anti-CTLA4 antibodies and/or anti-PD-1 antibodies. In an exemplary embodiment the LIGHT-containing conjugate is administered prior to the one or more immune checkpoint inhibitors.

In a further particular embodiment the method comprises administration of the LIGHT polypeptide conjugated to a tumour homing peptide, optionally an RGR-containing peptide, in combination with one or more immune checkpoint inhibitors and a tumour-specific vaccine. In an exemplary embodiment the LIGHT-containing conjugate is administered prior to the one or more immune checkpoint inhibitors and the tumour-specific vaccine.

A fourth aspect of the invention provides a method for increasing or extending the survival time of a cancer patient, the method comprising administering to the subject an effective amount of a peptide-protein conjugate comprising a LIGHT polypeptide and a tumour homing peptide.

The peptide-protein conjugate may be administered to the subject in combination with chemotherapy, immunotherapy and/or radiotherapy. The conjugate may be administered to the subject prior to, concomitantly with, or subsequent to the chemotherapy, immunotherapy and/or radiotherapy.

Immunotherapy may comprise adoptive cell transfer or the administration of one or more anti-tumour or immune checkpoint inhibitors, tumour-specific vaccines or other immune cell modulating agents optionally with, for example, autologous tumour material or known anti-tumour antigen/adjuvant formulations. Adoptive cell transfer may comprise the transfer of autologous tumour infiltrating lymphocytes. In exemplary embodiments, the immune checkpoint inhibitor may comprise anti-CTLA4 antibodies or anti-PD-1 antibodies. In exemplary embodiments the chemotherapy may comprise administration of cyclophosphamide.

In a particular embodiment the method comprises administration of the LIGHT polypeptide conjugated to a tumour homing peptide, optionally an RGR-containing peptide, in combination with one or more immune checkpoint inhibitors. The one or more immune checkpoint inhibitors may comprise anti-CTLA4 antibodies and/or anti-PD-1 antibodies. In an exemplary embodiment the LIGHT-containing conjugate is administered prior to the one or more immune checkpoint inhibitors.

In a further particular embodiment the method comprises administration of the LIGHT polypeptide conjugated to a tumour homing peptide, optionally an RGR-containing peptide, in combination with one or more immune checkpoint inhibitors and a tumour-specific vaccine. In an exemplary embodiment the LIGHT-containing conjugate is administered prior to the one or more immune checkpoint inhibitors and the tumour-specific vaccine.

In accordance with the third and fourth aspects, the effective amount of the peptide-protein conjugate may be between about 6 ng per kg body weight and about 600 to 700 ng per kg body weight.

A fifth aspect of the invention provides a method for increasing the sensitivity of a tumour to chemotherapy, immunotherapy and/or radiotherapy, the method comprising exposing the tumour to an effective amount of a peptide-protein conjugate comprising a LIGHT polypeptide and a tumour homing peptide.

The tumour may be resistant to one or more chemotherapeutic agents, immunotherapeutic agents or radiotherapeutic agents in the absence of said treatment.

A sixth aspect of the invention provides a peptide-protein conjugate comprising a LIGHT polypeptide and a tumour homing RGR-containing peptide.

A seventh aspect of the invention provides a pharmaceutical composition comprising a LIGHT-RGR conjugate according to the sixth aspect.

Also provided is a polynucleotide encoding a peptide-protein conjugate according to the sixth aspect.

Also provided is the use of a peptide-protein conjugate comprising a LIGHT polypeptide and a tumour homing peptide in the manufacture of a medicament for normalizing tumour vasculature and stroma and/or improving vascular function in a tumour, for treating a tumour or for increasing the survival time of a patient with a tumour.

### BRIEF DESCRIPTION OF THE DRAWINGS

Embodiments of the invention are described herein, by way of non-limiting example only, with reference to the following figures.

FIG. 1. Schematic illustration of short term treatment of RIP1-Tag5 mice as exemplified herein.

FIG. 2. Schematic illustration of long term treatment of RIP1-Tag5 mice as exemplified herein.

FIG. 3. LIGHT targeted to the tumour microenvironment normalizes the tumour vasculature. 0.2 ng LIGHT (which corresponds to a dose of 6-7 ng/kg) or LIGHT-RGR was injected i.v. bi-weekly for 2 weeks and tissues analyzed by histology. A-B. LIGHT-RGR induced a significant increase in small vessels (<30 μm in size) and a decrease in larger vessels (150-200 μm) while the total surface area of CD31 remained intact (B, right). C. LIGHT-RGR significantly decreased the protrusion of αSMA+ pericytes from the vasculature and decreased stromal Col IV, not associated with the vasculature. Note the lining of vascular basement membrane Col IV staining in close proximity to the normalized vasculature.

FIG. 4. LIGHT-RGR improves vascular function and tumour perfusion. Following 2 week treatment with LIGHT or LIGHT-RGR, 70 kDa TRITC-Dextran or FITC-Lectin was injected i.v. The tissues were perfused with formalin and embedded in OCT. Intra tumoural levels of Dextran and Lectin respectively was analyzed using a Nikon Ti-E microscope and quantified by using NIS software (version 3.0). 0.2 ng LIGHT-RGR reduced vascular leakiness and increased tumour perfusion. In the graphs, left hand bars=0.2 ng LIGHT (n=5); right hand bars=0.2 ng LIGHT-RGR (n=4). * p=<0.05.

FIG. 5. LIGHT-RGR induces a pericyte switch into a more contractile phenotype. 25 wk old RIP1-Tag5 mice were biweekly injected i.v. 0.2 ng LIGHT-RGR and tissues collected for histology. A. Immunohistochemical analysis of the vascular marker CD31 with the contractile markers Calponin and Caldesmon in relation to CD31 and αSMA. Note, both Calponin and Caldesmon were found exclusively in αSMA positive pericytes associated with the tumour vasculature. B. Calponin and Caldesmon were found to be significantly up regulated following LIGHT-RGR treatment (right hand bars) versus controls (left hand bars). * p=<0.05.

FIG. 6. LIGHT-RGR activates the tumour vasculature and enhances T cell infiltration after short term treatment. A. LIGHT-RGR treatment (0.2 ng, 2 bi-weekly injections for 2 weeks) increases expression of the inflammatory adhesion molecule ICAM-1 on tumour ECs. B. Control and 0.2 ng LIGHT-RGR treated mice were injected i.v. with in vitro activated CD8+ T cells and tumours harvested and analysed for CD8+ T cells. 0.2 ng LIGHT-RGR significantly improves T cell infiltration compared to either treatment on their own. Scale bars, A. 100 μm, B. 50 * p=<0.05.

FIG. 7. LIGHT-RGR prolongs survival in adoptive transfer and vaccine combination therapies. A. Mice were treated with adoptive transfer of in vitro activated CD8+ T cells and LIGHT-RGR and survival assessed at a set endpoint (30 weeks). 0.2 ng LIGHT-RGR treated tumours after 2 adoptive transfers (2×AdT) were pale which is indicative of increased vascular function and immune cell infiltration. P=0.038 (Fisher's exact/Pearson's Chi-square test). B. Mice were vaccinated with anti-Tag protein and treated with LIGHT or LIGHT-RGR and survival monitored. P=0.006 vaccine+LR compared to vaccine only.

FIG. 8. 0.2 ng LIGHT-RGR and chemotherapy. Mice were treated long term with LIGHT (control) or LIGHT-RGR bi-weekly i.v. combined with low dose cyclophosphamide in drinking water. A. Intratumoural apoptosis (TUNEL) was analysed by histology in different treatment groups and B. quantified. **p=<0.01, n=5-7 mice. C. Assessment of tumour burden after treatment. *p=0.02 compared to untreated, **p≤0.001 compared to all experimental groups, n=10-12 mice. Scale bar, 100 μm D. Survival analysis. RIP1-Tag5 mice were treated from week 22 with LIGHT-RGR, cyclophosphamid and a combination. Survival was monitored. P=0.05 cyclophosphamide+LR compared to cyclophosphamide alone, n=8-10 mice.

FIG. 9. Ectopic lymph node structures containing high endothelial venules (HEVs) in tumours of RIP1-Tag5 mice treated for two weeks with biweekly i.v. injections of 20 ng LIGHT-RGR (which corresponds to a dose of 600-700 ng/kg). HEVs were observed in 60-75% of treated tumours. Top: HEV structures are visualized with MECA79 antibodies (red), green (lectin) depicts vessels. Middle: HEV structures are associated with infiltrating immune cells (CD45+). Bottom: similar to lymph node structures, B cells (B220) are in the centre of the immune infiltrate.

FIG. 10. Tumour cell depletion after long term treatment of RIP1-Tag5 mice with 20 ng LIGHT-RGR. Dapi-staining (A, upper panel) and H&E stained (B) tumour sections show a substantial reduction of tumour cells. Increase in TUNEL signal shows an increase in tumour cell apoptosis (A, lower panel).

FIG. 11. Survival data following treatment with 20 ng LIGHT-RGR in RIP1-Tag5 mice in combination with checkpoint blockade and anti-tumour vaccination. A. RIP1-Tag5 mouse survival with LIGHT-RGR and anti-PD1/CTLA4 antibody treatment from week 23 to 45. B. 20 ng LIGHT-RGR and anti-Tag vaccine+/−antibodies. For A and B, n=10-12; *P<0.001, **P<0.0001 to untreated.

FIG. 12. Short-term (two week) treatment in 26-week-old RIP1-Tag5 mice as indicated. Tumours were isolated and total tumour burden determined by weight. LR=20 ng LIGHT-RGR. Statistical significance indicated. N=mouse numbers.

FIG. 13. LIGHT-RGR induces vessel normalization in murine breast cancer, which in turn increases vessel perfusion and reduces tumor hypoxia. Mice with orthotopically implanted 4T1 breast cancer were treated for 2 weeks with 20 ng LIGHT-RGR i.v. A. Assessment of overall vascularity (CD31+ vessels). B. Analysis of perfusion with FITC-lectin. C. Induction of contractile marker caldesmon after treatment. D. Assessment of tumor hypoxia after pimonidazole injection. N=3-6 mice, scale bars, A, 100 B-D, 50 μm. Left hand images=untreated. Right hand images=LIGHT-RGR treated.

FIG. 14. Binding of FAM labelled CREKA- and CGKRK-containing peptides to Panc02 pancreatic adenocarcinoma and Lewis lung carcinoma, respectively. Visualization was of FAM-labelled peptides with anti-FITC-HRP antibodies. Magnification: 40×.

A listing of amino acid and nucleotide sequences corresponding to the sequence identifiers referred to in the specification is provided. The amino acid sequences of human and mouse LIGHT are provided in SEQ ID NOs:1 and 3, respectively. The nucleotide sequences of human and mouse LIGHT are provided in SEQ ID NOs:2 and 4, respectively. SEQ ID NO:5 provides the amino acid sequence of the exemplified RGR-containing peptide used in the present studies, while SEQ ID NO:6 provides the amino acid sequence of the exemplified LIGHT-RGR conjugate used in the present studies. Sequences of further exemplary tumour homing peptides are provided in SEQ ID Nos:7 to 13 and 15.

### DETAILED DESCRIPTION

The articles “a” and “an” are used herein to refer to one or to more than one (i.e., to at least one) of the grammatical object of the article. By way of example, “an element” means one element or more than one element.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

In the context of this specification, the term “about,” is understood to refer to a range of numbers that a person of skill in the art would consider equivalent to the recited value in the context of achieving the same function or result.

As used herein the term “LIGHT” refers to the polypeptide designated “homologous to lymphotoxins, exhibits indicible expression, and competes with HSV glycoprotein D for HVEM, a receptor expressed by T lymphocytes”. LIGHT binds to the herpes virus entry mediator (HVEM) and to the lymphotoxin β receptor (LTβR). LIGHT is also known as TNFSF14.

The term “polypeptide” means a polymer made up of amino acids linked together by peptide bonds. The term “peptide” may also be used to refer to such a polymer although in some instances a peptide may be shorter (i.e. composed of fewer amino acid residues) than a polypeptide. The terms “polypeptide” and “protein” may be used interchangeably herein.

The term “tumour homing peptide” as used herein refers to a peptide that has the ability to recognise and bind tumour cells, typically tumour vasculature or stromal cells. Thus the term “tumour homing peptide” may be used interchangeably with “tumour vasculature homing peptide”. Such recognition and binding may be preferential, specific or selective for the tumour, tumour vasculature or stromal cells.

As used herein the terms “treating” and “treatment” and grammatical equivalents refer to any and all uses which remedy a tumour, prevent the establishment of a tumour, or otherwise prevent, hinder, retard, or reverse the progression of a tumour. Thus the term “treating” is to be considered in its broadest context. For example, treatment does not necessarily imply that a patient is treated until total recovery.

As used herein the term “effective amount” includes within its meaning a non-toxic but sufficient amount or dose of an agent or compound to provide the desired effect. The exact amount or dose required will vary from subject to subject depending on factors such as the species being treated, the age, size, weight and general condition of the subject, the severity of the tumour being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

As used herein the term “sensitivity” is used in its broadest context to refer to the ability of a cell to survive exposure to an agent designed to inhibit the growth of the cell, kill the cell or inhibit one or more cellular functions.

As used herein the term “resistance” is used in its broadest context to refer to the reduced effectiveness of a therapeutic agent to inhibit the growth of a cell, kill a cell or inhibit one or more cellular functions, and to the ability of a cell to survive exposure to an agent designed to inhibit the growth of the cell, kill the cell or inhibit one or more cellular functions. The resistance displayed by a cell may be acquired, for example by prior exposure to the agent, or may be inherent or innate. The resistance displayed by a cell may be complete in that the agent is rendered completely ineffective against the cell, or may be partial in that the effectiveness of the agent is reduced.

The term “subject” as used herein refers to mammals and includes humans, primates, livestock animals (e.g. sheep, pigs, cattle, horses, donkeys), laboratory test animals (e.g. mice, rabbits, rats, guinea pigs), performance and show animals (e.g. horses, livestock, dogs, cats), companion animals (e.g. dogs, cats) and captive wild animals. Preferably, the mammal is human or a laboratory test animal. Even more preferably, the mammal is a human.

As described and exemplified herein the inventors have identified therapeutic utilities for peptide-protein conjugates comprising the LIGHT polypeptide and a tumour homing peptide against tumours. Exemplified herein is, inter alia, the ability of such conjugates to manipulate tumour stromal cells and normalize the vasculature of tumours, both structurally and functionally, to induce high endothelial venules (HEVs) in tumours, to kill tumour cells and to increase the survival of subjects bearing tumours. Exemplified peptide-protein conjugates are also shown to sensitize tumour cells to chemotherapeutic agents, including agents to which the tumour may otherwise display resistance. Moreover the ability of exemplified peptide-protein conjugates to extend survival time of tumour-bearing subjects, in particular when administered in conjunction with one or more immunotherapeutic agents, is also exemplified.

Without wishing to be bound by theory, the inventors suggest that the peptide-protein conjugates defined herein directly stimulate intratumoral stromal cells, and as a result of this, indirectly act to normalise tumour vessels. For example, the inventors have found that macrophages in the tumour secrete TGFβ after LIGHT-RGR stimulation, which normalizes vessels. For HEV induction, LIGHT-RGR stimulates stromal cells to secrete CCL21, which is most likely responsible for inducing HEVs.

In one aspect the invention described herein provides a method for modulating tumour stroma, normalizing tumour vasculature and/or improving vascular function in a tumour, the method comprising exposing a tumour to an effective amount of a peptide-protein conjugate comprising a LIGHT polypeptide and a tumour homing peptide.

The normalization of tumour vasculature and improvement of vessel function may be determined, assessed or measured by a number of means or parameters well known to those skilled in the art. By way of example only, normalization of tumour vasculature and improvement of vessel function may comprise or be characterized by one or more of: differential secretion of factors/cytokines by stromal cells including but not limited to vascular cells, selective loss of large vessels; reduced leakiness of vessels; pericyte re-attachment to vessels; alignment of surrounding collagen IV fibres; enhanced infiltration of CD8+ and/or CD45+ T cells; increased expression of inflammatory adhesion molecules; increased expression of contractile markers in a smooth muscle (αSMC)-positive tumour pericytes, and/or phenotype switching of pericytes from a dedifferentiated state to a differentiated state.

Normalization of tumour vasculature and/or improvement of tumour vessel function may comprise or result in one or more of restoration of tumour blood vessel integrity, a reduction in leakiness of tumour blood vessels and/or an increase in tumour perfusion. As a result, methods and compositions of the present invention find application in reducing edema formation associated with tumours such as, for example, brain tumours, and find application in reducing metastatic spreading of tumours, more particularly reducing blood borne tumour metastasis.

In another aspect the invention provides a method for inducing the formation of ectopic lymph node structures harbouring high endothelial venules (HEVs) in a tumour, the method comprising exposing the tumour to an effective amount of a peptide-protein conjugate comprising a LIGHT polypeptide and a tumour homing peptide.

In a further aspect the invention provides a method for treating a tumour in a subject, the method comprising administering to the subject an effective amount of a peptide-protein conjugate comprising a LIGHT polypeptide and a tumour homing peptide.

In a further aspect the invention provides a method for increasing or extending the survival time of a cancer patient, the method comprising administering to the subject an effective amount of a peptide-protein conjugate comprising a LIGHT polypeptide and a tumour homing peptide.

In a further aspect the invention provides a method for increasing the sensitivity of a tumour to a chemotherapeutic agent, immunotherapeutic agent or radiotherapeutic agent by improving tumour perfusion, the method comprising exposing the tumour to an effective amount of a peptide-protein conjugate comprising a LIGHT polypeptide and a tumour homing peptide.

Particular clinical embodiments of the invention contemplate the administration of a ‘low dose’ (optionally between about 0.2 to 20 ng per kg body weight) of the protein conjugate as a tumour vessel normalization agent, optionally to be used in combination with immunotherapy (such as adoptive cell transfer, vaccination, or vaccination plus immune-checkpoint control) or chemotherapy or both. The conjugate may be used as an adjuvant to facilitate access of immune cells or drugs into tumours. The contemplated ‘low dose’ treatment does not induce destruction of tumour stromal (support) cells (which the present inventors have analyzed for up to 8 weeks continuous treatment; data not shown), which is often seen with long term treatments of existing anti-angiogenic and chemotherapeutic drugs. Destruction of stroma (including vessels) by existing anti-angiogenic drugs may have initial beneficial anti-tumour effects, but ultimately causes relapse.

Particular clinical embodiments of the invention contemplate the administration of a ‘high dose’ (optionally between about 20 to 2000 ng per kg body weight) of the peptide-protein conjugate alone or as an adjuvant with immune stimulation (such as adoptive cell transfer, vaccination, checkpoint control inhibitors or vaccination plus checkpoint control inhibitors). The conjugate may be use to facilitate access of adaptive immune cells to the tumour environment and create optimal conditions for anti-tumour T cell priming.

The LIGHT polypeptide to be used in peptide-protein conjugates in accordance with the present invention may comprise the amino acid sequence set forth in SEQ ID NO:1, representing the native human LIGHT sequence, encoded by the polynucleotide sequence set forth in SEQ ID NO:2. Homologues of human LIGHT may also be employed, including for example the mouse polypeptide with an amino acid sequence set forth in SEQ ID NO:3. Embodiments of the present invention also contemplate the employment of variants of LIGHT.

The term “variant” as used herein refers to substantially similar sequences. Generally, polypeptide sequence variants also possess qualitative biological activity in common, such as receptor binding activity. Further, these polypeptide sequence variants may share at least 50%, 55%, 60%, 65%, 70%, 75%, 80%, 85%, 90%, 91%, 92%, 93%, 94%, 95%, 96%, 97%, 98% or 99% sequence identity. The term “sequence identity or “percentage of sequence identity” may be determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences.

The tumour homing peptide for use in peptide-protein conjugates of the present invention may be any peptide capable of targeting or directing a LIGHT polypeptide to which it is conjugated to a tumour, or optionally to tumour vasculature or other stromal cells (e.g. macrophages, fibroblasts, other immune cells or extracellular matrix components). Suitable peptides capable of such homing or targeting will be well known to those skilled in the art. Examples include peptides comprising the peptide motif RGR, NGR, CGKRK (SEQ ID NO:7), CREKA (SEQ ID NO:8), RGD, isoDGR, SRPRR (SEQ ID NO:9), CDTRL (SEQ ID NO:10), the HMGN2-derived peptides PQRRSARLSA (SEQ ID NO:11) or KDEPQRRSARLSAKPAPPKPEPKPKKAPAKK(SEQ ID NO:12), LyP-1 (CGNKRTRGC; SEQ ID NO:13), or conservative variants thereof. In particular embodiments of the present invention the homing peptide comprises the RGR peptide, such as, for example, the peptide CRGRRSTG (SEQ ID NO:5) (Joyce et al., 2003). However the skilled addressee will appreciate that the scope of the present invention is not limited to the exemplified homing peptide, and numerous other suitable peptides may be employed (see, for example, Li and Cho, 2012). Additionally, recognizing that the binding affinity of different homing peptides may vary depending on the specific tumour, those skilled in the art will appreciate that it represents mere optimization to determine the most appropriate homing peptide to employ in any given circumstance. For example, the present inventors have determined that in at least some settings, as determined by semi-quantitative immunohistochemistry, CREKA-containing peptides bind strongly to panc02 pancreatic adenocarcinomas in mice while CGKRK-containing peptides home preferentially to Lewis lung cell carcinomas in mice (data not shown). Thus, data provided herein demonstrating activity and efficacy of LIGHT conjugated to an RGR-containing peptide are provided by way of exemplification only.

Tumour homing peptides for use in accordance with the invention can have, for example, a relatively short length of less than four, five, six, seven, eight, nine, ten, 12, 15, 20, 25, 30, 35, 40, 45, 50, 60, 70 or 80 residues, typically as a contiguous sequence. Alternatively, the peptide may retain homing activity when provided in the context of (e.g. embedded in) a larger peptide, polypeptide or protein sequence. Thus, the invention further provides chimeric peptides, polypeptides and proteins containing a tumour homing peptide fused to a heterologous peptide, polypeptide or protein. Such a chimeric peptide, polypeptide or protein may have a length of, for example, up to about 10, 15, 20, 25, 30, 35, 40, 45, 50, 55, 60, 65, 70, 75, 80, 85, 90, 95, 100, 150, 200, 250, 300, 350, 400, 450, 500, 800, 1000 or 2000 residues or more.

Peptidomimetics of tumour homing peptides are also contemplated and encompassed by the present disclosure. The term “peptidomimetic,” as used herein means a peptide-like molecule that has the tumour homing activity of the peptide upon which it is structurally based. Such peptidomimetics include chemically modified peptides, peptide-like molecules containing non-naturally occurring amino acids, and peptoids (see, for example, Goodman and Ro, Peptidomimetics for Drug Design, in “Burger's Medicinal Chemistry and Drug Discovery” Vol. 1 (ed. M. E. Wolff; John Wiley & Sons 1995), pages 803-861).

A variety of peptidomimetics are known in the art including, for example, peptide-like molecules which contain a constrained amino acid (for example an α-methylated amino acid, α,α-dialkyl glycine, α-, β- or γ-aminocycloalkane carboxylic acid, an α,β-unsatruated amino acid, a β,β-dimethyl or β-methyl amino acid or other amino acid mimetic), a non-peptide component that mimics peptide secondary structure (for example a nonpeptidic 3-turn mimic, γ-turn mimic, a mimic of β sheet structure, or a mimic of helical structure), or an amide bond isostere (for example a reduced amide bond, methylene ether bond, ethylene bond, thioamide bond or other amide isostere). Methods for identifying peptidomimetics are also well known in the art and include, for example, the screening of databases that contain libraries of potential peptidomimetics.

Tumour homing peptides or peptidomimetcis of the invention may be cyclic or otherwise conformationally constrained. Conformationally constrained molecules can have improved properties such as increased affinity, metabolic stability, membrane permeability or solubility. Methods of conformational constraint are well known in the art.

The tumour homing peptide may be conjugated to the N-terminal or C-terminal end of the LIGHT polypeptide. The conjugate may or may not include a short linker sequence between the LIGHT polypeptide and the homing peptide. In exemplary embodiments the linker comprises one or more, optionally two or more or three or more glycine (G) residues.

Also provided herein are peptide-protein conjugates per se, comprising a LIGHT polypeptide and a tumour homing peptide, optionally an RGR-containing peptide. In exemplary embodiments the conjugate may comprise the LIGHT polypeptide sequence as set forth in SEQ ID NO:1 or 3 and the RGR-containing peptide sequence as set forth in SEQ ID NO:5, or may comprise the amino acid sequence set forth in SEQ ID NO:6. Also provided are nucleotide sequences comprising peptide-protein conjugates disclosed and contemplated herein.

Any suitable amount or dose of a peptide-protein conjugate may be administered to a subject in need in accordance with the present invention. The therapeutically effective amount for any particular subject may depend upon a variety of factors including: the tumour being treated and the severity of the tumour; the activity of the conjugate employed; the composition employed; the age, body weight, general health, sex and diet of the subject; the time of administration; the route of administration; the rate of sequestration of the molecule or agent; the duration of the treatment; drugs used in combination or coincidental with the treatment, together with other related factors well known in medicine. One skilled in the art would be able, by routine experimentation, to determine an effective, non-toxic amount of protein conjugate to be employed.

The effective amount of peptide-protein conjugate may be between about 0.1 ng per kg body weight to about 100 μg per kg body weight, or typically between about 0.2 ng per kg body weight and about 10 μg per kg body weight. The effective amount may be, for example, about 0.2 ng, 0.4 ng, 0.6 ng, 0.8 ng, 1 ng, 1.5 ng, 2 ng, 2.5 ng, 3 ng, 3.5 ng, 4 ng, 4.5 ng, 5 ng, 5.5 ng, 6 ng, 6.5 ng, 7 ng, 7.5 ng, 8 ng, 8.5 ng, 9 ng, 9.5 ng, 10 ng, 11 ng, 12 ng, 13 ng, 14 ng, 15 ng, 16 ng, 17 ng, 18 ng, 19 ng, 20 ng, 25 ng, 30 ng, 35 ng, 40 ng, 45 ng, 50 ng, 55 ng, 60 ng, 65 ng, 70 ng, 75 ng, 80 ng, 85 ng, 90 ng, 95 ng, 100 ng, 150 ng, 200 ng, 250 ng, 300 ng, 350 ng, 400 ng, 450 ng, 500 ng, 550 ng, 600 ng, 650 ng, 700 ng, 750 ng, 800 ng, 850 ng, 900 ng, 950 ng, 1000 ng, 1100 ng, 1200 ng, 1300 ng, 1400 ng, 1500 ng, 1600 ng, 1700 ng, 1800 ng, 1900 ng or 2000 ng per kg body weight. As noted above, in particular embodiments ‘low dose’ and ‘high dose’ treatment, of between about 0.2 to 20 ng per kg body weight and between about 20 to 2000 ng per kg body weight, respectively, with the protein conjugate are contemplated for use in specific scenarios. Very high dose of about, or above 6 to 7 μg per kg body weight can be considered if vessel death is a desired outcome.

The skilled addressee will appreciate that among the factors determining the appropriate dose of conjugate to be administered will be the nature of the tumour homing peptide employed and the affinity, selectivity and/or specificity of that tumour homing peptide for the particular tumour type to be treated.

The skilled addressee will also recognise that in determining an appropriate and effective dosage range for administration to humans based on the mouse studies exemplified herein, dose escalation studies would be conducted. The skilled addressee would therefore appreciate that the above mentioned doses and dosage ranges are exemplary only based on the doses administered in the mouse studies exemplified herein, and the actual dose or dosage range to be employed in humans may be varied depending on the results of such dose escalation studies. Based on the data exemplified herein, the appropriate and effective dose or dosage range to be administered to humans can be determined by routine optimisation, without undue burden or experimentation.

By virtue of the ability of homing peptides disclosed herein to target tumor vasculature, the methods and compositions disclosed herein are applicable to the treatment of any cancerous tumour, including, but not limited to, those associated with: lung cancer, including small cell lung cancer and non-small cell lung cancer; pancreatic cancer, including insulinomas; bladder cancer; kidney cancer; breast cancer; brain cancer, including glioblastomas and medulloblastomas; neuroblastoma; head and neck cancer; thyroid cancer; cervical cancer; prostate cancer; testicular cancer; ovarian cancer; endometrial cancer; rectal and colorectal cancer; stomach cancer; esophageal cancer; skin cancer, including melanomas and squamous cell carcinomas; oral cancer including squamous cell carcinoma; liver cancer, including human hepatocellular carcinona (HCC); lymphomas; sarcomas, including osteosarcomas, liposarcomas and fibrosarcomas.

Particular embodiments disclosed herein contemplate combination treatments, wherein administration of the peptide-protein conjugate is in conjunction with one or more additional anti-tumour therapies. Such additional therapies may include, for example, radiotherapy, chemotherapy or immunotherapy/immune stimulation/deletion of stromal immune cells known to foster tumour growth, such as myeloid suppressor cells and regulatory T cells. Contemplated herein are synergistic combinations in which the combination treatment is effective in inhibiting growth, or reducing viability, of tumour cells, or increasing survival of subjects having tumours, to a greater extent than either component of the combination alone. Thus, in some embodiments a synergistically effective amount of a peptide-protein conjugate and, for example, a chemotherapeutic agent or immunotherapeutic agent is administered to a subject. A synergistically effective amount refers to an amount of each component which, in combination, is effective in inhibiting growth, or reducing viability, of cancer cells, and which produces a response greater than either component alone.

For such combination therapies, each component of the combination therapy may be administered at the same time, or sequentially in any order, or at different times, so as to provide the desired effect. Alternatively, the components may be formulated together in a single dosage unit as a combination product. When administered separately, it may be preferred for the components to be administered by the same route of administration, although it is not necessary for this to be so.

Suitable chemotherapeutic agents may be, for example, alkylating agents (such as cyclophosphamide, oxaliplatin, carboplatin, chloambucil, mechloethamine and melphalan), antimetabolites (such as methotrexate, fludarabione and folate antagonists) or alkaloids and other antitumour agents (such as vinca alkaloids, taxanes, camptothecin, doxorubicin, daunorubicin, idarubicin and mitoxantrone). In an exemplary embodiment the chemotherapeutic agent is an alkylating agent, optionally cyclophosphamide.

Immunotherapy or immune stimulation may comprise, by way of example only, adoptive cell transfer or the administration of one or more anti-tumour or immune checkpoint inhibitors, tumour-specific vaccines or immune-cell depleting reagents. Adoptive cell transfer typically comprises the recovery of immune cells, typically T lymphocytes from a subject and introduction of these cells into a subject having a tumour to be treated. The cells for adoptive cell transfer may be derived from the tumour-bearing subject to be treated (autologous) or may be derived from a different subject (heterologous).

Suitable immune checkpoint inhibitors include antibodies such as monoclonal antibodies, small molecules, peptides, oligonucleotides, mRNA therapeutics, bispecfic/trispecific/multispecific antibodies, domain antibodies, antibody fragments thereof, and other antibody-like molecules (such as nanobodies, affibodies, T and B cells, ImmTACs, Dual-Affinity Re-Targeting (DART) (antibody-like) bispecific therapeutic proteins, Anticalin (antibody-like) therapeutic proteins, Avimer (antibody-like) protein technology), against immune checkpoint pathways. Exemplary immune checkpoint antibodies include anti-CTLA4 antibodies (such as ipilimumab and tremelimumab), anti-PD-1 antibodies (such as MDX-1106 [also known as BMS-936558], MK3475, CT-011 and AMP-224), and antibodies against PDL1 (PD-1 ligand), LAG3 (lymphocyte activation gene 3), TIM3 (T cell membrane protein 3), B7-H3 and B7-H4 (see, for example, Pardoll, 2012). However these are provided by way of example only, and those skilled in the art will appreciate that other antibodies directed to T cells or antibodies directed to other tumour cell markers may be employed. The identity of suitable anti-tumour antibodies will depend, for example, on the nature or type of tumour to be treated. Suitable anti-tumour antibodies will be well known to those skilled in the art (see, for example, Ross et al., 2003). Cells for adoptive cell transfer and anti-tumour or immune checkpoint antibodies may be regarded, collectively, as immunotherapy agents.

In a particular embodiment the invention provides a method for increasing or extending the survival time of a subject having a tumour, comprising administering a LIGHT polypeptide conjugated to a tumour homing peptide, optionally an RGR-containing peptide, in combination with one or more immune checkpoint inhibitors. The one or more immune checkpoint inhibitors may comprise anti-CTLA4 antibodies and/or anti-PD-1 antibodies. In an exemplary embodiment the LIGHT-containing conjugate is administered prior to the one or more immune checkpoint inhibitors.

In a particular embodiment the invention provides a method for increasing or extending the survival time of a subject having a tumour, comprising administering a LIGHT polypeptide conjugated to a tumour homing peptide, optionally an RGR-containing peptide, in combination with one or more immune checkpoint inhibitors and a tumour-specific vaccine. In an exemplary embodiment the LIGHT-containing conjugate is administered prior to the one or more immune checkpoint inhibitors and the tumour-specific vaccine.

Particular embodiments disclosed herein contemplate the sensitization of tumours and tumour cells to chemotherapeutic agents, immunotherapy agents and radiotherapeutic agents using protein conjugates as disclosed herein. The tumour or tumour cells may display resistance to the chemotherapeutic agent, immunotherapy agent or radiotherapeutic agent in the absence of treatment with the protein conjugate.

Embodiments of the present invention also therefore provide methods for determining a change in sensitivity of a tumour or tumour cell to a chemotherapeutic agent, immunotherapy agent or radiotherapeutic agent. Such methods may comprise

(a) administering to a subject a protein conjugate comprising a LIGHT polypeptide and a tumour homing peptide;
(b) determining the sensitivity or resistance to the agent in a biological sample from the subject comprising at least one tumour cell;
(c) repeating steps (a) and (b) at least once over a period of time; and
(d) comparing said sensitivity or resistance in the samples.

Exposure of tumours to peptide-protein conjugates as defined herein to modulate tumour stroma, normalize tumour vasculature, improve vessel function, induce HEVs, sensitize the tumour to a chemotherapeutic or immunotherapeutic agent, or otherwise treat the tumour, may comprise administering or targeting the conjugate to a vascular component and/or a stromal component of the tumour, and to tumour cells and/or extracellular matrix.

Protein conjugates as disclosed herein may be administered to subjects, or contacted with cells, in accordance with aspects and embodiments of the present invention in the form of pharmaceutical compositions, which compositions may comprise one or more pharmaceutically acceptable carriers, excipients or diluents suitable for in vivo administration to subjects, and optionally one or more chemotherapeutic, immunotherapeutic and/or radiotherapeutic agents. Where multiple agents are to be administered, for example in synergistic combinations as disclosed herein, each agent in the combination may be formulated into separate compositions or may be co-formulated into a single composition. If formulated in different compositions the compositions may be co-administered. By “co-administered” is meant simultaneous administration in the same formulation or in two different formulations via the same or different routes or sequential administration by the same or different routes. By “sequential” administration is meant a time difference of from seconds, minutes, hours or days between the administration of the two compositions. The compositions may be administered in any order, although in particular embodiments it may be advantageous for the peptide-protein conjugate to be administered prior to the chemotherapeutic, immunotherapeutic or radiotherapeutic agent.

Compositions may be administered to subjects in need thereof via any convenient or suitable route such as by parenteral (including, for example, intraarterial, intravenous, intramuscular, subcutaneous), topical (including dermal, transdermal, subcutaneous, etc), oral, nasal, mucosal (including sublingual), or intracavitary routes. Thus compositions may be formulated in a variety of forms including solutions, suspensions, emulsions, and solid forms and are typically formulated so as to be suitable for the chosen route of administration, for example as an injectable formulations suitable for parenteral administration, capsules, tablets, caplets, elixirs for oral ingestion, in an aerosol form suitable for administration by inhalation (such as by intranasal inhalation or oral inhalation), or ointments, creams, gels, or lotions suitable for topical administration. The preferred route of administration will depend on a number of factors including the tumour to be treated and the desired outcome.

The most advantageous route for any given circumstance can be determined by those skilled in the art. For example, in circumstances where it is required that appropriate concentrations of the desired agent are delivered directly to the site in the body to be treated, administration may be regional rather than systemic. Regional administration provides the capability of delivering very high local concentrations of the desired agent to the required site and thus is suitable for achieving the desired therapeutic or preventative effect whilst avoiding exposure of other organs of the body to the compound and thereby potentially reducing side effects.

In general, suitable compositions may be prepared according to methods known to those of ordinary skill in the art and may include a pharmaceutically acceptable diluent, adjuvant and/or excipient. The diluents, adjuvants and excipients must be “acceptable” in terms of being compatible with the other ingredients of the composition, and not deleterious to the recipient thereof. Pharmaceutical carriers for preparation of pharmaceutical compositions are well known in the art, as set out in textbooks such as Remington's Pharmaceutical Sciences, 20^{th}Edition, Williams& Wilkins, Pa., USA. The carrier will depend on the route of administration, and again the person skilled in the art will readily be able to determine the most suitable formulation for each particular case.

For administration as an injectable solution or suspension, non-toxic parenteral acceptable diluents or carriers can include Ringer's solution, medium chain triglyceride (MCT), isotonic saline, phosphate buffered saline, ethanol and 1,2 propylene glycol. Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emollients, emulsifiers, thickening agents, preservatives, bactericides and buffering agents.

Methods for preparing parenteral administrable compositions are apparent to those skilled in the art, and are described in more detail in, for example, Remington's Pharmaceutical Science, 15th ed., Mack Publishing Company, Easton, Pa., hereby incorporated by reference herein. The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silica ceoussilicas, and other ingredients such as lanolin, may also be included.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include com starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propylparaben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate. Polyoxyethylene sorbitan mono- or di-oleate, -stearate or -laurate and the like.

Emulsions for oral administration may further comprise one or more emulsifying agents. Suitable emulsifying agents include dispersing agents as exemplified above or natural gums such as guar gum, gum acacia or gum tragacanth.

Compositions of the invention may be packaged and delivered in suitable delivery vehicles which may serve to target or deliver the peptide-protein conjugate, and optionally one or more additional agents to the required tumour site and/or to facilitate monitoring of tumour uptake by, for example MRI imaging or other imaging techniques known in the art. By way of example, the delivery vehicle may comprise liposomes, or other liposome-like compositions such as micelles (e.g. polymeric micelles), lipoprotein-based drug carriers, microparticles, nanoparticles, or dendrimers.

Liposomes may be derived from phospholipids or other lipid substances, and are formed by mono- or multi-lamellar hydrated liquid crystals dispersed in aqueous medium. Specific examples of liposomes used in administering or delivering a composition to target cells are DODMA, synthetic cholesterol, DSPC, PEG-cDMA, DLinDMA, or any other non-toxic, physiologically acceptable and metabolisable lipid capable of forming liposomes. The compositions in liposome form may contain stabilisers, preservatives and/or excipients. Methods for preparing liposomes are well known in the art, for example see Methods in Cell Biology, Volume XIV, Academic Press, New York, N.Y. (1976), p. 33 ff., the contents of which are incorporated herein by reference. Biodegradable microparticles or nanoparticles formed from, for example, polylactide (PLA), polylactide-co-glycolide (PLGA), and epsilon-caprolactone (\acute{ε}-caprolactone) may be used.

Other means of packaging an/or delivering peptide-protein conjugates, and optionally one or more additional agents, in order to monitor tumour uptake will also be well known to those skilled in the art.

Embodiments of the invention described herein employ, unless otherwise indicated, conventional molecular biology and pharmacology known to, and within the ordinary skill of, those skilled the art. Such techniques are described in, for example, “Molecular Cloning: A Laboratory Manual”, 2^{nd} Ed., (ed. by Sambrook, Fritsch and Maniatis) (Cold Spring Harbor Laboratory Press: 1989); “Nucleic Acid Hybridization”, (Hames& Higgins eds. 1984); Oligonucleotide Synthesis” (Gait ed, 1984); Remington's Pharmaceutical Sciences, 17^{th} Edition, Mack Publishing Company, Easton, Pa., USA.; “The Merck Index”, 12th Edition (1996), Therapeutic Category and Biological Activity Index, and “Transcription & Translation”, (Hames & Higgins eds. 1984).

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to the following specific examples, which should not be construed as in any way limiting the scope of the invention.

### EXAMPLES

### Experimental Procedures

### Mice

RIP1-Tag5 transgenic mice were used on a C3HeBFe background (provided by D. Hanahan, ISREC, Lausanne, Switzerland). For adoptive transfer experiments, mice transgenic for a T cell receptor (TCR) that recognizes Tag presented by the MEW class I molecule H-2Kk (referred to as TagTCR8; provided by T. Geiger, St. Jude Children's Research Hospital, Memphis, Tenn., USA and R. Flavell, Yale University, New Haven, Conn., USA) were used on a C3H background. All mice were kept under specific pathogen-free conditions at the University of Western Australia and all experimental protocols were approved by the Animal Ethics Committee of the University of Western Australia.

### Production of Recombinant LIGHT (L) and LIGHT-RGR (LR)

Mature murine LIGHT (SEQ ID NO:3), with or without a C-terminal modified RGR peptide CRGRRSTG (SEQ ID NO:5) connected via a GGG linker (LIGHT-RGR conjugate—SEQ ID NO:6) were cloned into Xho/BamH1 sites of the vector pET-44a (Novagen) to express soluble fusion proteins with N-terminal Nus⋅Tag/His⋅Tag. Briefly, after isopropyl-β-d-glactopyranoside (IPTG) induction for 6 hours at 22° C. in the presence of 5 mM EGTA, cultures were centrifuged, resuspended in lysis buffer (50 mM NaH2PO4, 300 mM NaCl, 10 mM Imidazole, 1 mM DTT, 1 mM PMSF, 1 mM EDTA/EGTA, 1% Triton-X100, 1× Protease inhibitor cocktail (Sigma), 1 ug/ml pepstatin (Calbiochem), (pH 8.0), followed by sonication, and subsequent purification using Ni-NTA beads (Qiagen) following the manufacturer's instructions. The recombinant fusion protein was dialysed in Tris buffer (50 mM Tris, 1 mM EDTA/EGTA, 1 mM PMSF), pH 8.0, overnight at 4° C. Nus⋅Tag/His⋅Tag was cleaved with tobacco etch virus (TEV) protease (Life Technologies) for 2 hours at 30° C. Fully active LIGHT protein from the cleavage reactions were re-purified using Ni-NTA beads in the presence of protease inhibitors (1 mM PMSF, 1 mM EDTA/EGTA, 1 ug/ml Pepstatin and 1× Protease inhibitor cocktail (Sigma)), salts (50 mM NaH₂PO₄, 300 mM NaCl, 10 mM Imidazole) and 0.005% BSA. Purity was assessed on Coomassie brilliant blue stained protein gels and the concentration was determined by measuring the intensity of the band compared to a band of similar size, and known concentration.

### Treatment of Tumour-Bearing RIP1-Tag5 Mice

RIP1-Tag5 mice were treated as follows:

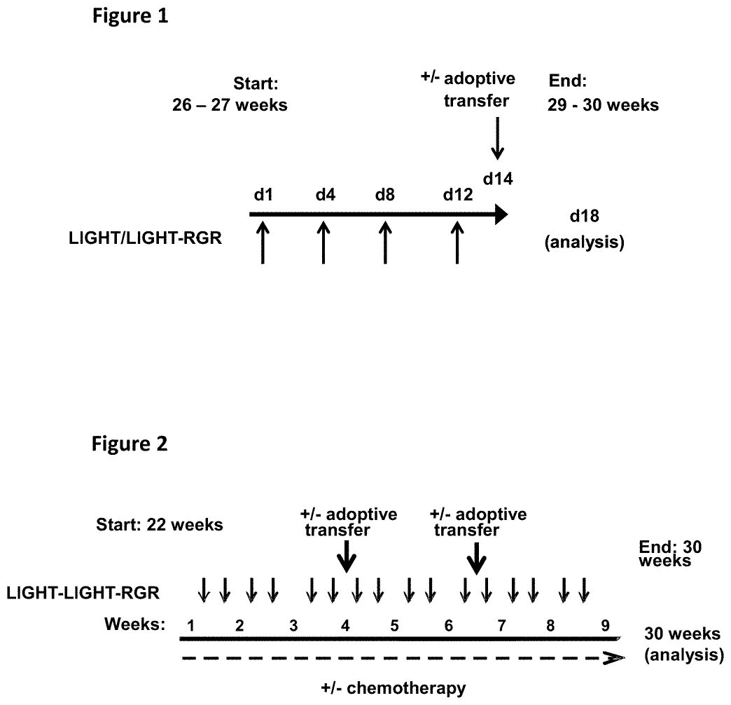

Short term treatment: commencing at 26-27 weeks of age mice were treated for 2 weeks with bi-weekly intravenous (i.v.) injections of recombinant proteins in 100 μl volume at 0.2 ng (equivalent to approximately 0.0006 ng/g body weight for a 30 g mouse) or 20 ng (equivalent to approximately 600-700 ng/g body weight for a 30 g mouse) LIGHT or LIGHT-RGR. When indicated this was followed by one adoptive transfer of TagTCR8 (CD8+) T cells. Four days later, mice were sacrificed and tumours were isolated for histology. For adoptive transfer experiments, TagTCR8 lymph node cells were activated in vitro for 3 days, with 10 U of rIL-2/ml and 25 nM Tag peptide 362-568 (SEFLIEKRI). 2.5×10⁶ activated CD8⁺ T cells were injected i.v once. The short term treatment regimen is shown schematically in FIG. 1

Long Term Treatment and Survival Studies:

22 week-old RIP1-Tag5 mice were treated with bi-weekly i.v. injections of recombinant proteins as described for short term regimens for a total of 8 weeks and survival/tumour burden analysed at the set endpoint of 30 weeks. For adoptive transfer experiments, 2.5×10⁶ activated TagTCR8 CD8⁺ T cells were injected i.v twice (at week 4 and week 6). In adoptive transfer experiments (without LIGHT-RGR), mice received two adoptive transfers only. Chemotherapy: 22 week-old mice were treated with i.v. injections of 0.2 ng LIGHT-RGR bi-weekly over a period of 8 weeks and were simultaneously provided with 20 mg/ml cyclophosphamide (metronomic low dose) in drinking water throughout the experiment. The long term treatment regimen is shown schematically in FIG. 2. In addition, mice were treated with cyclophosphamide and/or LIGHT-RGR and survival monitored (death as endpoint).

For vaccination, mice were primed with one subcutaneous injection (tail base) of 50 μg recombinant Tag protein mixed with 50 μg Freund's adjuvant (Sigma). Thereafter, cytosine-phosphorothioate-guanine oligodeoxynucleotide (CpG-ODN) treatment groups were injected with 50 μg recombinant Tag protein mixed with 50 μg CpG-ODN 1668 (TCCATGACGTTCCTGATGCT; SEQ ID NO:14) i.p. every second week, as previously published (Garbi et al, 2004). For combination therapies with checkpoint blockade, RIP1-Tag5 mice were treated with LIGHT-RGR (20 ng, iv, biweekly) in combination with anti-PD1 (250 i.p.) and anti-CTLA4 (75 i.p.) antibodies (BioXCell). In addition, mice were treated with triple combination of LIGHT-RGR/anti-PD1+anti CTL4/anti-Tag vaccine.

### Histology

Mice were perfused with 2% neutral-buffered formalin before removal of tumours. Tumours were incubated in 10% (2 h) and 30% sucrose overnight and embedded in OCT compound. For immunohistochemistry the following antibodies were used: anti B220 (BD Pharmingen), anti-CD8 (Ly-2, BD Pharmingen), anti-CD31 (Mec 13.3., BD Pharmingen), anti-ICAM2 (3C4, BD Pharmigen), anti-CD45 (30-F11, BD Pharmingen), anti-CD68 (FA-11, BD Biosciences), anti-calponin (rabbit monoclonal EP798Y, Abcam), anti-caldesmon (rabbit monoclonal E89, Abcam), anti-collagen I or collagen IV (rabbit polyclonal, Abcam), Ki67 (PP67, Abcam), MECA79 (American type tissue culture, ATCC) and αSMA (1A4, Sigma). For secondary detection, AMCA (7-Amino-4-methylcoumarin-3-acetic acid), Cy-3 or FITC-conjugated IgG F(ab′)2 fragments (Jackson Immuno Research) were used. The αSMA staining was amplified using the mouse on mouse (M.O.M.) kit (Vector). For lectin perfusion, mice were i.v. injected with 50 μg of FITC-labelled tomato lectin (Lycopersicon esculentum, Vector). After 10 min of circulation, mice were heart-perfused with 2% neutral-buffered formalin and tumours frozen in OCT. To evaluate vessel leakiness, 1 mg of 70 kDa Texas Red Dextran (Invitrogen) was injected i.v. and allowed to circulate for 10 min. Mice were heart-perfused with PBS followed by 2% neutral buffered formalin and tumours frozen in OCT. Apoptosis was assessed using TUNEL staining (Roche). Images were recorded on a Nikon Ti-E microscope and quantified using NIS software modules (version 3.0).

### Breast Tumour Model

Murine breast cancer cells (5×10⁶, 4T1 from ATCC) were injected orthotopically into the mammary fat pad of Balb/c mice. After tumours became palpable, mice were treated for 2 weeks with bi-weekly injections of 20 ng LIGHT-RGR i.v. Mice were injected with pimonidazole (hypoxia marker) and FITC-labelled lectin. After 1 h/10 min (pimonidazole/lectin, respectively) circulation, mice were perfused with 2% formalin and tumors dissected and fresh frozen in OCT compound. Tumours were analyzed by histology for vessel frequency (CD31), quality of vessel perfusion (CD31 plus lectin-FITC), caldesmon induction and frequency of intratumoral hypoxia (pimonidazole staining).

### Statistics

Cumulative survival time was calculated by the Kaplan-Meier method and analyzed by the log-rank test. Student's t test (2-tailed) was used unless indicated otherwise. A P value of less than 0.05 was considered statistically significant. Error bars indicate SD unless stated otherwise.

### RIP1-Tag5 Mouse Model

To study the complex interrelationship between tumour, tumour vessels and the immune system, the inventors' analyses focused on a transgenic mouse model which develops spontaneous tumours that mimic the clinical scenario with regard to native anatomical location, slow growth kinetics, and multistep tumour progression. In RIP1-Tag5 mice, the oncogene SV40 Large T antigen (Tag; RIP, rat insulin gene promoter) is exclusively expressed in β cells of the pancreas leading to stepwise tumour development through well-characterized stages progressing from hyperplastic islets, to the initiation of Tag expression at approximately 10 weeks, the onset of blood vessel formation in angiogenic islets (termed “angiogenic switch”) at approximately 16 weeks and then to highly vascularised solid tumours at approximately 22 weeks, followed by death at approximately 30 weeks.

### Example 1—Short Term and Long Term Treatment of RIP1-Tag5 Mice with 0.2 ng LIGHT-RGR

### Short Term Treatment

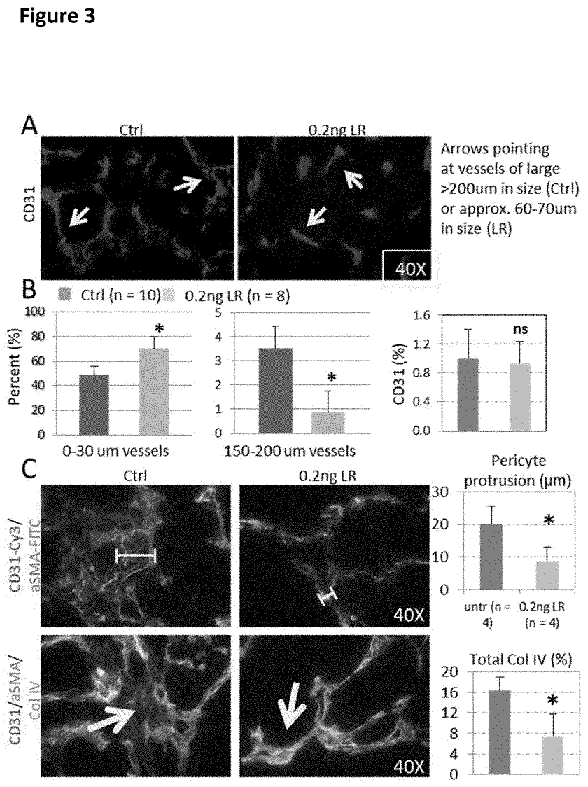

0.2 ng LIGHT-RGR when injected a total of four times into tumour-bearing RIP1-Tag5 mice normalized chaotic tumour vessels (FIG. 3) as determined histologically.

As shown in FIGS. 3A and 3B, a shift from large to small caliber tumour vessels (i.e. a selective loss of large vessels) was observed, without affecting total vessel counts (as determined using CD31 as a vessel marker).

Pericytes are support cells that wrap around endothelial cells of the blood vessels. A classical feature of chaotic tumour vessels is the protrusion of pericytes into the tumour parenchyma (see FIG. 3C). In contrast, firm attachment of pericytes to vessels was observed in LIGHT-RGR treated tumours (FIG. 3C). This pericyte re-attachment to vessels was accompanied by close vessel alignment of collagen IV, which also indicates normalization of the vascular bed, in addition to improved endothelial cell/pericyte alignment (FIG. 3C).

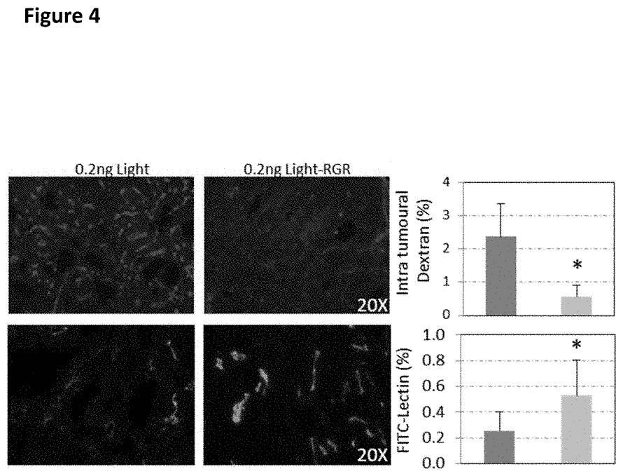

Injection of 0.2 ng LIGHT-RGR also improved the function of vessels within the tumour. Tumour vasculature is characteristically “leaky”. This can be demonstrated by extravasation of red-labelled dextran. In the present study tumours treated with LIGHT alone produced a leaky phenotype, whereas LIGHT-RGR treatment normalized tumour vessels and produced a less leaky phenotype (FIG. 4). I.v. injection of a FITC-labelled lectin was also demonstrated to stain tumour vessels green, indicative of adequate perfusion, in tumours treated with LIGHT-RGR. Green staining was not observed in untreated, chaotic tumour vessels (FIG. 4).

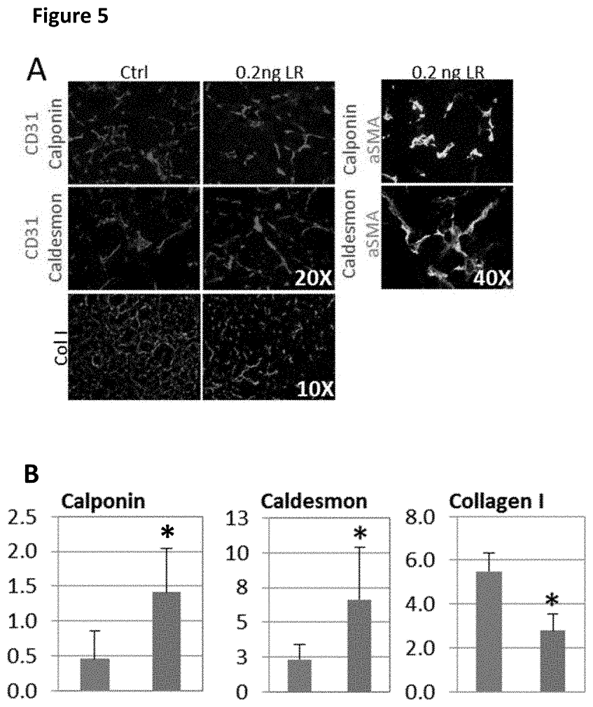

Short term treatment with 0.2 ng LIGHT-RGR also resulted in a significant increase in expression of “contractile” vascular smooth muscle markers including calponin and caldesmon in a smooth muscle (αSMC)-positive tumour pericytes (FIG. 5). In contrast, the expression of collagen I was significantly down regulated in normalized vessels (FIG. 5). Collagen I is a synthetic marker and contractile cells secrete less collagen I.

This is a remarkable finding as it represents the first demonstration of a phenotypic switch in pericytes upon normalization. These data show that pericytes in normalized vessels change from a “dedifferentiated” into a “differentiated” state upon treatment with LIGHT-RGR.

The inventors have also demonstrated (data not shown) that LIGHT-RGR stimulates macrophages in the tumour environment to secrete TGFβ. TGFβ at low dose released in the vicinity of the vessels causes the pericyte phenotype switch. Briefly, this was demonstrated by: isolating tumour resident macrophages from LIGHT or LIGHT-RGR treated tumours; collecting supernatant from ex vivo purified macrophages (determining TGFβ secretion with ELISA, specific for LIGHT-RGR treated macrophages); incubating supernatant from macrophages with a pericyte cell line in vitro; and determining calponin/caldesmin expression in vitro which can be blocked by anti-TGFβ blocking antibodies. Calponin/caldesmin are not induced with direct LIGHT induction of pericyte cells (data not shown).

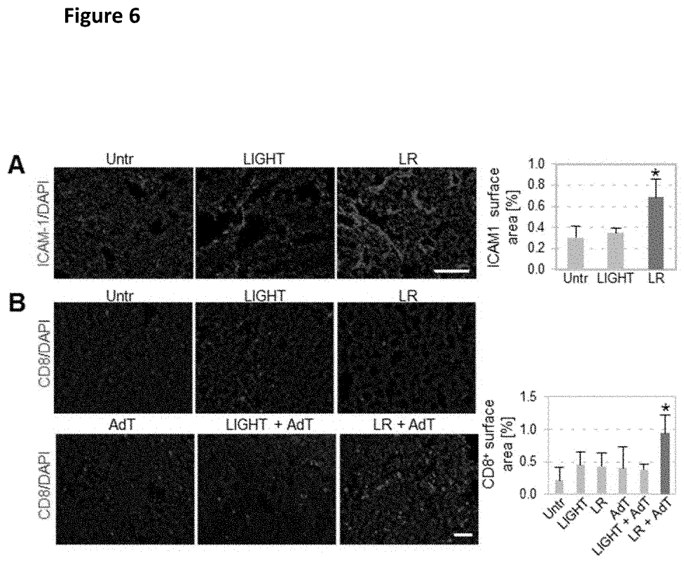

Short term treatment with 0.2 ng LIGHT-RGR was also shown to increase expression of the inflammatory adhesion molecule ICAM-1 on tumour endothelial cells (FIG. 6A) and enhance CD8+ T cell infiltration. Normalized vessels, induced by LIGHT-RGR treatment, allowed increased transmigration of adoptively transferred CD8+ T cells (FIG. 6B). 0.2 ng LIGHT-RGR treatment in combination with adoptive transfer of in vitro activated CD8+ T cells significantly improved T cell infiltration compared to either treatment on their own (FIG. 6B).

### Long Term Treatment

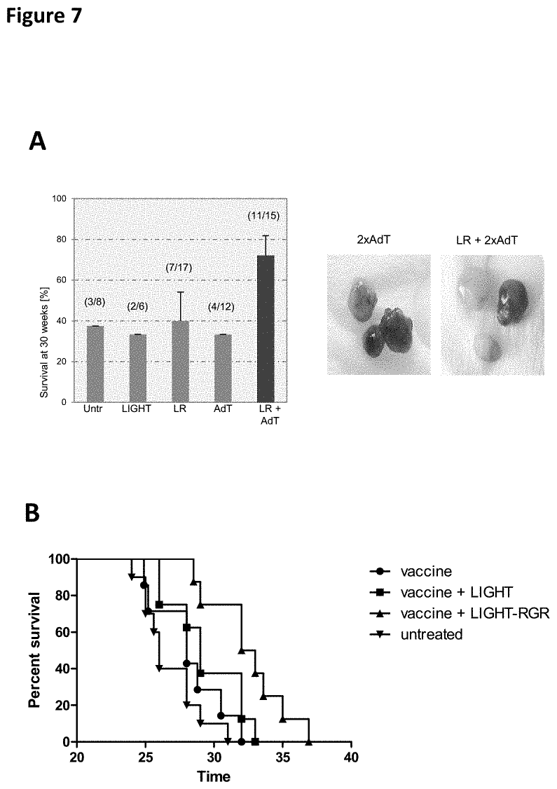

Long term treatment of tumours in RIP1-Tag5 mice with 0.2 ng LIGHT-RGR, in combination with adoptive transfer of anti-tumour (anti-Tag) lymphocytes (in vitro activated) led to a substantial inflammatory response at the tumour site. Macroscopically: this was observed by a change in tumour appearance, from highly hemorrhagic, red and leaky tumours to tumours with normalized vessels with a white appearance (FIG. 7A). Adoptive T cell transfer or 0.2 ng LIGHT-RGR as single treatments resulted in approximately 30% survival of RIP1-Tag5 mice at week 30, whereas the combination of both treatment modalities enhanced survival to approximately 70% at week 30 (FIG. 7A). This result is indicative that the enhanced T cell influx depicted in FIG. 6 indeed increases survival of tumour-bearing mice.

In a further survival study RIP1-Tag5 mice were vaccinated with anti-Tag protein and treated with 0.2 ng LIGHT-RGR or LIGHT alone. As shown in FIG. 7B, treatment with LIGHT-RGR in combination with anti-Tag vaccination substantially increased survival over vaccination alone or vaccination in combination with LIGHT.

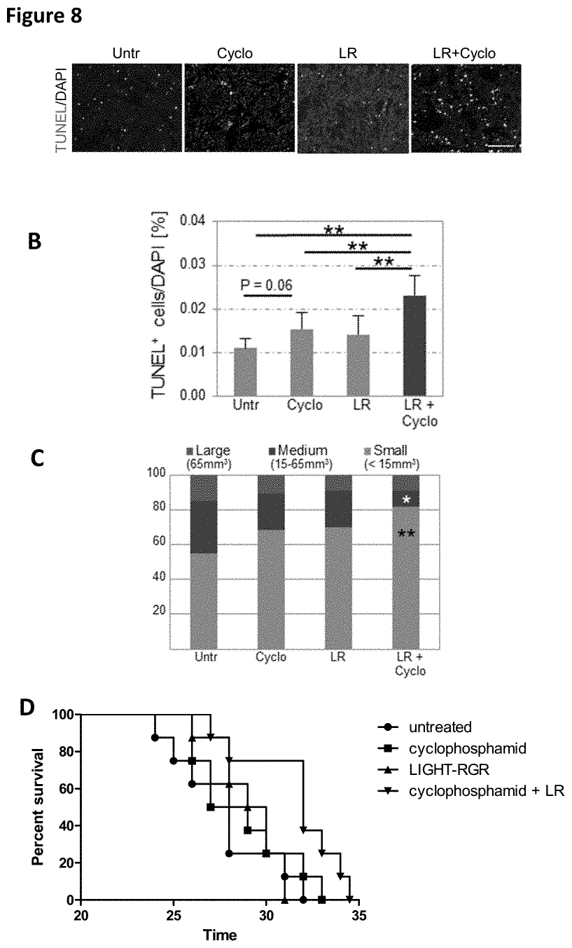

A combination of treatment with 0.2 ng LIGHT-RGR and low dose, metronomic chemotherapy comprising cyclophosphamide in drinking water was shown to improve tumour cell killing as evidenced by a significant increase in apoptotic tumour cells after 8 weeks of treatment (FIGS. 8 A and B) compared to each treatment alone. This is also reflected in a shift from large tumours to smaller tumours after 8 weeks of LIGHT-RGR/cyclophosphamide combination treatment (FIG. 8C). These results demonstrate that, with tumour stroma manipulation and vessel normalization due to LIGHT-RGR treatment, cytotoxic drugs are able to reach the tumour and kill tumour cells. RIP1-Tag tumours (insulinomas) are normally unresponsive to metronomic cyclophosphamide treatment. Survival analysis (FIG. 8D) demonstrated a significant increased survival of RIP1-Tag5 mice treated from week 22 with LIGHT-RGR in combination with cyclophosphamide compared to either treatment alone.

The inventors also studied the effects of injection of 2 ng LIGHT-RGR into RIP1-Tag5 tumours and found similar effects to those observed with 0.2 ng (data not shown). Injection of 2 μg caused vessel death in RIP1-Tag5 mice.

### Example 2—Short Term and Long Term Treatment of RIP1-Tag 5 Mice with 20 ng LIGHT-RGR

### Short Term Treatment

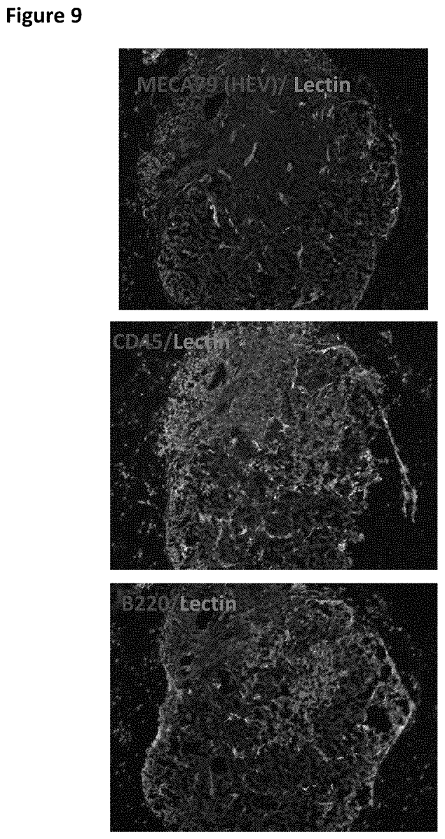

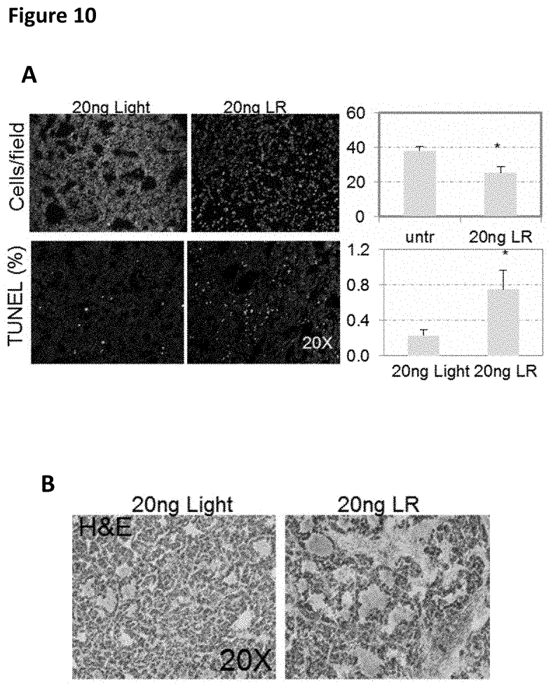

Short term treatment of tumours in RIP1-Tag5 mice with 20 ng LIGHT-RGR induced the formation of ectopic lymph node structures (CD45/B220+ lymphocytes, FIG. 9) associated with high endothelial venules (HEVs) as recognized immunohistochemically by the marker MECA79 (FIG. 9). HEVs serve as portals for the mass transit of lymphocytes in and out of activated lymph nodes and heavily inflamed tissues. This is the first demonstration of HEV formation in tumours in response to a single therapeutic agent.

Specifically, following short term treatment with 20 ng LIGHT-RGR HEV structures were observed in 60-75% of RIP1-Tag5 tumours, with intratumoural areas surrounding the HEVs heavily infiltrated with T cells and B cells. Interestingly, T cells which infiltrate tumors comprise CD4+ and CD8+ populations which also includes PD-1+ and CTLA4+ T cells and regulatory T cells as analyzed by FACS (data not shown). This provides the rational to combine induction of ectopic lymph nodes with checkpoint blockade to improve T cell priming and function associated with these structures.

### Long Term Treatment

Following long term treatment with 20 ng LIGHT-RGR, RIP1-Tag5 tumours were found to be highly necrotic at 30 weeks with a substantial reduction of tumour cells (FIG. 10). The observed increase in TUNEL signals is indicative of an increase in tumour cell apoptosis, consistent with anti-tumour effects of 20 ng LIGHT-RGR.

The inventors then extensively tested the effect of immunotherapy (using anti-PD-1 and ant-CTLA4 monoclonal antibodies) in conjunction with 20 ng LIGHT-RGR in a cohort of >120 RIP1-Tag5 mice. RIP1-Tag5 mice were treated with LIGHT-RGR (20 ng, iv, biweekly) in combination with anti-PD-1 (250 μg) and anti-CTLA4 (75 μg) antibodies (BioXCell). In addition, mice were treated with triple combination of LIGHT-RGR/anti-PD1+anti CTL4/anti-Tag vaccine.

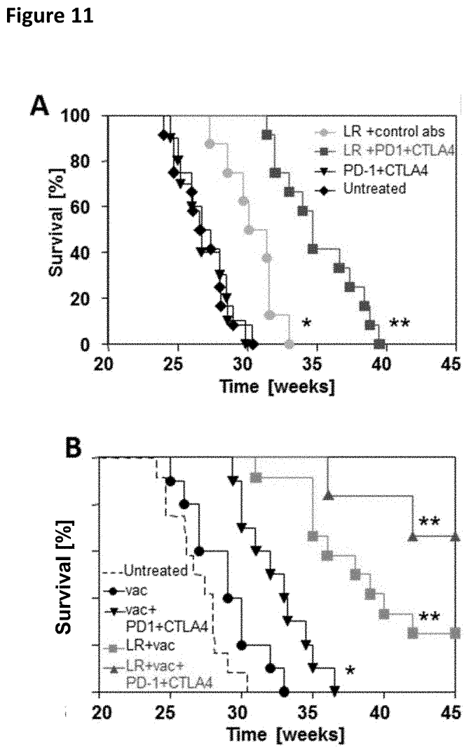

In survival studies, the inventors have shown that the triple combination of LIGHT-RGR/anti-PD-1/anti-CTLA4 significantly increased survival (P<0.0001 compared to controls; FIG. 11A). LIGHT-RGR treatment with tumour specific vaccine results in 30% survival at 45 weeks of age (normal life span 26-32 weeks) (FIG. 11B). Significantly improved survival results were obtained with LIGHT-RGR+vaccine+anti-PD-1+anti-CTLA4, with 70% of RIP1-Tag5 mice alive at 45 weeks (FIG. 11B). This survival advantage is mediated by LIGHT-RGR pre-treatment of tumours, which validates intra-tumoural LIGHT effects as an adjuvant to immunotherapy.

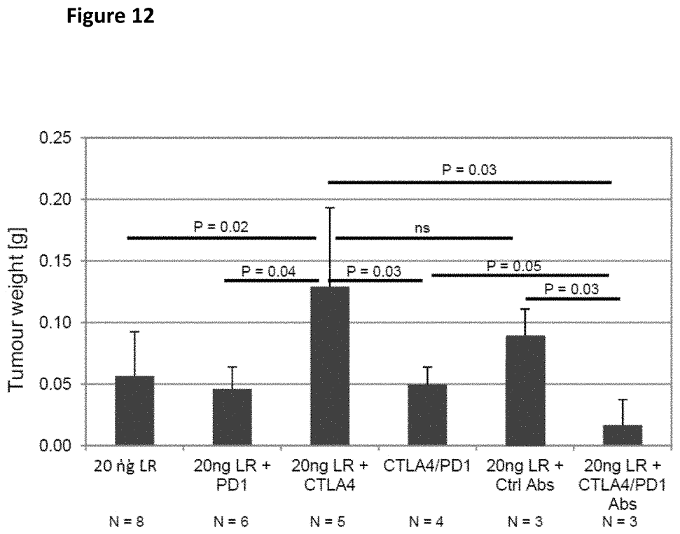

In a short term (two week) experiment the inventors also showed that the efficacy of anti-PD-1/anti-CTLA4 double treatment in combination with LIGHT-RGR was superior to single treatment with the respective anti-PD-1 and anti-CTLA4 monoclonal antibodies (FIG. 12).

### Example 3—Effect of LIGHT-RGR on Murine Breast Cancer Tissue Vasculature

Murine breast cancer cells (5×10⁶, 4T1 from ATCC) were injected orthotopically into the mammary fat pad of Balb/c mice. After tumours became palpable, mice were treated for 2 weeks with bi-weekly injections of 20 ng LIGHT-RGR i.v. Mice were injected with pimonidazole (hypoxia marker) and FITC-labelled lectin. After 1 h/10 min (pimonidazole/lectin, respectively) circulation, mice were perfused with 2% formalin and tumors dissected and fresh frozen in OCT compound. Tumours were analyzed by histology for vessel frequency (CD31), quality of vessel perfusion (CD31 plus lectin-FITC), caldesmon (contractile pericyte marker) induction and frequency of intratumoral hypoxia (pimonidazole staining). 0.2 ng LIGHT-RGR reproduced all aspects of vessel normalization as shown for RIP1-Tag5 mice. However in the breast cancer model, due to lower binding affinity to tumor vessels, a dose of 20 ng LIGHT-RGR was required to recapitulate the vessel phenotype of RIP1-Tag5 mice treated with 0.2 ng.

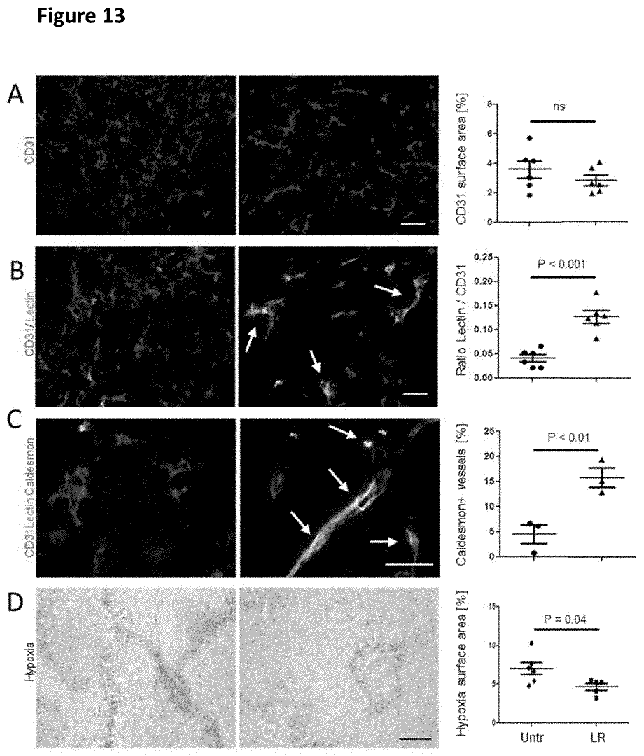

As shown in FIG. 13, 20 ng LIGHT-RGR treatment induced vessel normalization (as evidenced by reduced vessel calibers without change in overall vascularity; FIG. 13A), increased vessel perfusion (FIG. 13B), induced contractile markers in pericytes (exemplified by Caldesmon induction, FIG. 13C) and reduced tumour hypoxia (FIG. 13D).

### Example 4—Binding of Tumour Homing Peptides to Different Tumour Types

The inventors tested the ability of different vasculature homing peptides to bind different tumour types. RGR- and NGR-containing peptides, and CGKRK- and CREKA peptides were tested against B16 melanoma, Lewis lung carcinoma, 4T1 breast carcinoma and orthotopic panc02 pancreatic cancer in mice. Peptides were labelled with FAM to enable detection by immunohistochemistry.

FAM-labelled, linear peptides were synthesized (Auspep Pty Ltd) with C-terminus amidation and 6-aminohexanoic acid spacer. The NGR-containing peptide used was CNGRCG (SEQ ID NO:15). 100 μg of FAM-peptide was injected i.v. into tumour-bearing mice (see below). After 30 min of circulation, tumours were collected and fresh frozen tissue sections further analysed with anti-FITC HRP antibodies to quantify vascular binding.

1×10⁶ panc02 tumour cells (pancreatic adenocarcinoma, ATCC) were injected in 30 μl in a PBS/matrigel mix into the pancreas of C57BL/6 mice (survival surgery). After 4 weeks, mice were injected with FAM-labelled peptide and sacrificed for intra-tumoural analysis. Lewis lung tumour cells (1×10⁶, LL2, ATCC) were inoculated subcutaneously and mice injected with peptides at day 10.

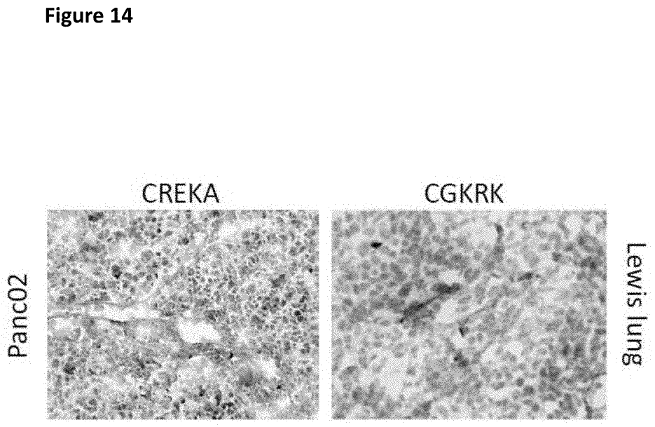

All vascular homing peptides tested were found to bind to blood vessels in all tumour models tested. Binding strength (as assessed by semi-quantitative immunohistochemistry) differed between tumour models. By way of example only, in the experiments conducted the CREKA-containing peptide bound strongly to panc02 tumours, whereas the CGKRK-containing peptide bound strongly to Lewis lung carcinoma cells (FIG. 14). Thus, different homing peptides have different binding affinities depending on tumour type.

### REFERENCES

Bergers, G., et al., Effects of angiogenesis inhibitors on multistage carcinogenesis in mice. Science, 1999. 284(5415): p. 808-12.
Ganss, R., et al., Combination of T-cell therapy and trigger of inflammation induces remodeling of the vasculature and tumour eradication. Cancer Res, 2002. 62(5): p. 1462-70.
Garbi, N., et al., CpG motifs as proinflammatory factors render autochthonous tumours permissive for infiltration and destruction. J Immunol, 2004. 172(10): p. 5861-9.
Geiger, T., L. R. Gooding, and R. A. Flavell, T-cell responsiveness to an oncogenic peripheral protein and spontaneous autoimmunity in transgenic mice. Proc Natl Acad Sci USA, 1992. 89(7): p. 2985-9.
Joyce, J. A., et al., Stage-specific vascular markers revealed by phage display in a mouse model of pancreatic islet tumourigenesis. Cancer Cell, 2003. 4(5): p. 393-403.
Li, Z. J. and Ho C. H. Peptides as targeting probes against tumour vasculature for diagnosis and drug delivery. J Trans Med, 2012, 10(Suppl 1):S1.
Pardoll, D. M. The blockade of immune checkpoints in cancer immunotherapy. Nature Reviews, 2012, 12:252-264.
Ross, J. S. et al. Anticancer antibodies. Am J Clin Pathol, 2003. 119:472-485.

1-20. (canceled)

21. A method for treating a tumour in a subject, comprising selecting a subject having a tumour and administering to said subject an effective amount of a peptide-protein conjugate comprising a LIGHT polypeptide and a tumour homing peptide selected from an RGR-containing peptide, a CGKRK (SEQ ID NO:7)-containing peptide or a CREKA (SEQ ID NO:8)-containing peptide, wherein the LIGHT polypeptide and the tumour-homing peptide are joined by a single linker, and wherein the conjugate normalizes tumour vasculature when contacted with the tumour vasculature.

22. The method of claim 21, wherein said subject is selected as one in need of improved tumor vascular function.

23. The method of claim 21, wherein said subject is selected as one in need of improved tumor perfusion.

24. The method of claim 21, wherein said subject is selected as one in need of enhanced T cell infiltration of said tumor.

25. The method of claim 21, wherein said subject is selected as one in need of an increased survival time.

26. The method of claim 21, wherein said subject is further provided a chemotherapeutic agent, checkpoint inhibitor, immunotherapy, or radiotherapy.

27. The method of claim 26, wherein peptide-protein conjugate is administered to the subject prior to, concomitantly with, or subsequent to the chemotherapy, immunotherapy and/or radiotherapy.

28. The method of claim 26, wherein said immunotherapy comprises the administration of one or more immune checkpoint inhibitors.

29. The method of claim 28, wherein said one or more immune checkpoint inhibitors comprise anti-CTLA4 antibodies and/or anti-PD-1 antibodies.

30. The method of claim 21, wherein an improvement of vessel function is measured.

31. The method of claim 30, wherein the improvement of vessel function is measured by assessing selective loss of large vessels, reduced leakiness of vessels, pericyte reattachment to vessels, alignment of surrounding collagen IV fibres, enhanced infiltration of CD8+ and/or CD45+ T cells, increased expression of inflammatory adhesion molecules, increased expression of contractile markers in alpha smooth muscle-positive pericytes, or phenotype switching of pericytes from a dedifferentiated state to a differentiated state.

32. The method of claim 21, wherein said LIGHT polypeptide comprises the amino acid sequence set forth in SEQ ID NO:1.

33. The method of claim 21, wherein said conjugate comprises a LIGHT polypeptide and an RGR-containing peptide, and wherein said RGR-containing peptide comprises the amino acid sequence set forth in SEQ ID NO:5.

34. A method for normalizing tumour vasculature in a subject having a tumour, comprising administering to said subject an effective amount of a peptide-protein conjugate comprising a LIGHT polypeptide and a tumour homing peptide selected from an RGR-containing peptide, a CGKRK (SEQ ID NO:7)-containing peptide or a CREKA (SEQ ID NO:8)-containing peptide, wherein the LIGHT polypeptide and the tumour-homing peptide are joined by a single linker, and wherein the conjugate normalizes tumour vasculature when contacted with the tumour vasculature.
